(19) United States
(12) Patent Application Publication
(10) Pub. No.: US 2023/0085259 A1
(43) Pub. Date: Mar. 16, 2023

(54) THE COMBINED USE OF PHYTASE AND PAD TO OBTAIN AN IMPROVED PLANT-BASED DRINK

(71) Applicant: DSM IP ASSETS B.V., Heerlen (NL)

(72) Inventors: Monica Diana VLASIE, Echt (NL); Christophe Joseph Etienne SCHMITT, Servion (CH); Johann BUCZKOWSKI, Corcelles-Le-Jorat (CH)

(21) Appl. No.: 17/906,826

(22) PCT Filed: Mar. 29, 2021
(86) PCT No.: PCT/EP2021/058142

§ 371 Date: Sep. 20, 2022

(30) Foreign Application Priority Data
Mar. 31, 2020 (EP) 20167167.4

(51) Int. Cl. A23J 3/34; A23J 3/14; A23L 2/66; A23L 11/60; A23L 11/30; C12N 9/16; C12N 9/78

(52) U.S. Cl. CPC A23J 3/346; A23J 3/14; A23L 2/66; A23L 11/60; A23L 11/33; C12N 9/16; C12Y 301/03008; C12N 9/78; C12Y 305/03015; A23V 2002/00

(57) ABSTRACT

The present invention relates to the food field. The invention provides a method for obtaining an improved property in a drink comprising a plant protein, said method comprising
incubating a solution comprising a plant protein with phytase and a peptidyl arginine deiminase (PAD) and
optionally processing the phytase and PAD treated solution into a drink comprising a plant protein, wherein the plant protein is pea protein or faba bean protein.

### FIELD

The present invention relates to the food field.

### BACKGROUND

In a world with a growing population them is an increased demand of proteins. To respond to this growing demand of proteins, there is a need to look at wider applications of proteins. In addition, it is desired to look for plant proteins as an alternative for animal proteins, since it is considered that plants are a more sustainable source of proteins than animals. The use of plant proteins in food is still limited in part because of poor texture and taste.

For example, drinks comprising pea protein often are astringent and have quite a pronounced off taste. This limits the expansion of a drink comprising pea protein in the market.

There is a need in the art to improve the properties of drinks comprising plant protein. The present patent application addresses this need.

### FIGURES

FIG. 1: The phosphorous compounds (IP6, IP5, IP1 and phosphate) in the pea flour ingredient without phytase enzyme treatment (flour and flour 30 min 50° C.) and with phytase enzyme treatment at 20 FTU/g dose (flour 30 min 50° C.+ phytase).

FIG. 2: The phosphorous compounds (IP6, IP5, IP1 and phosphate) in the pea isolate ingredient without phytase enzyme treatment (pea isolate and pea isolate 30 min 50° C.) and with phytase enzyme at 20 FTU/g treatment (pea isolate 30 min 50° C.+phytase).

### SUMMARY

The invention provides:
a method for preparing a plant protein comprising drink with an improved property, said method comprising
incubating a solution comprising a plant protein with phytase and a peptidyl arginine deiminase (PAD) (to obtain a phytase & PAD-treated solution) and
optionally processing the phytase and PAD treated solution into a drink comprising a plant protein;
a drink comprising plant protein obtainable by a method as described herein;
use of phytase and PAD for improving taste, texture and/or mouthfeel of a drink comprising a plant protein.

The plant protein may be, for example, pea protein or faba bean protein. Preferably, the plant protein is pea protein.

### DETAILED DESCRIPTION

At present, the texture and taste of plant protein-based food in general is obtained by adding stabilizers and thickeners such as hydrocolloids to improve texture or flavor to mask off flavors.

Surprisingly, the use of stabilizers, thickeners or flavors in a drink comprising plant protein can be reduced by using the enzymes peptidyl arginine deiminase (PAD) and phytase in the production process of plant-based drinks, particularly pea-based drinks or faba bean-based drinks. Preferably, the use of stabilizers, thickeners or flavors can be completely omitted such that a clean label product can be offered.

As a result, this invention results in a larger acceptation of a drink comprising plant protein due to taste and texture improvement. Minimally processed plant-based drinks increase acceptance and trust of consumers on plant-based foods alternatives to dairy products.

It is surprising that PAD and phytase can improve taste, texture, mouthfeel and/or viscosity in a drink comprising plant protein.

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

In one aspect, the invention provides a method for preparing a plant protein comprising drink with an improved property, said method comprising
incubating a solution comprising a plant protein with phytase and a peptidyl arginine deiminase (PAD) (to obtain a phytase & PAD-treated solution) and

optionally processing the phytase and PAD treated solution into a drink comprising a plant protein, wherein the plant protein is pea protein or faba bean protein.

Differently phrased, the invention provides a method for obtaining an improved property in a drink comprising a plant protein or a method for reducing the amount of off flavor masking compounds in a drink comprising plant protein or a method for reducing the amount of stabilizers and thickeners such as hydrocolloids in a drink comprising plant protein or a method for improving acceptance of a drink comprising plant protein or a method for improving taste, texture and/or mouthfeel in a drink comprising plant protein, any such method comprising
incubating a solution comprising a plant protein with phytase and a peptidyl arginine deiminase (PAD) (to obtain a phytase & PAD-treated solution) and

optionally processing the phytase and PAD treated solution into a drink comprising a plant protein, wherein the plant protein is pea protein or faba bean protein.

Preferably, the plant protein is pea protein.

The phrases “drink comprising plant protein” and “plant-based drink” and “plant protein drink” are used interchangeably herein. The plant protein may be, for example, pea protein or faba bean protein. Preferably, the plant protein is pea protein. For example, the phrases “drink comprising pea protein” and “pea drink” and “pea protein drink” refer to a drink which comprises at least 10% (based on al proteins present in said drink) pea protein. Preferably, a drink comprising pea protein comprises at least 20%, 30%, 40%, 50%, 60%, 70%, 80% or 90% (based on al proteins present in said drink) pea protein. Most preferably, a pea protein drink comprises only or exclusively (i.e. 100% based on al protein present in said drink) pea protein and no animal protein at al.

The phrases “solution comprising a plant protein” and “a plant protein comprising solution” are used interchangeably herein. The plant protein may be, for example, pea protein or faba bean protein. Preferably, the plant protein is pea protein. For example, the phrases “solution comprising a pea protein” and “a pea protein comprising solution” refer to a liquid composition which comprises a pea protein. Such a liquid composition is for example prepared by dissolving (or diluting depending on the state of matter of the starling material) a pea flour or pea protein concentrate or a pea protein isolate into a suitable liquid (for example water or a buffered solution). The concentration of pea protein in the pea protein comprising solution will for example depend on the desired drink (for example, a low protein or a high protein drink). The skilled person is able to prepare a solution comprising a pea protein with a suitable amount of pea protein. The concentration of pea protein in the pea protein solution is at least 0.5% (w/w), preferably at least 1% (w/w), more preferably at least 1.5% (w/w) and most preferred at least 2% (w/w). A typical suitable range of protein is 2 to 4% (w/w). Likewise for faba bean protein, the concentration of these proteins in the protein solution is at least 0.5% (w/w), preferably at least 1% (w/w), more preferably at least 1.5% (w/w) and most preferred at least 2% (w/w). A typical suitable range of protein is 2 to 4% (w/w).

Preferably, a method of the invention starts from a flour, protein concentrate or protein isolate. In case a flour, concentrate or isolate is not available, the skilled person can also start from, for example, peas and process them in a drink according to well-known methods which typically involve steps like (i) cleaning, soaking and/or dehulling, (ii) grinding of the starting material to produce a slurry, powder or emulsion, (iii) heating to inactivate endogenous enzymes, (iv) filtration to remove solids, (v) adding of water and optionally sugar, (v) pasteurization, (vii) homogenization and/or (viii) packaging, labeling and/or storage.

The preparation of a plant protein comprising solution may comprise mild (such as to avoid protein degradation) heating for a certain time and/or stirring for a certain time to ensure that the flour, protein concentrate or protein isolate is completely dissolved and a homogenous plant protein solution is obtained before the incubation with PAD and phytase takes place. Optionally, the pH can be adjusted by well-known methods such as the addition of an acid.

A method of the invention can thus comprise an additional step which comprises dissolving flour or powder to obtain a solution comprising a plant protein. Such a dissolving step typically comprises heating and/or stirring.

The “plant protein comprising solution” may be subjected to a heat treatment to reduce the amount of micro-organisms and to extend the shelf life of the final food product. Suitable heat treatments are pasteurization or ultra-high temperature (UHT) treatment.

The term“phytase” as used herein refers to an enzyme that is capable of breaking down phytic acid/phytate (inositol hexaphosphate), by hydrolyzing phytic acid/phytate so as to release at least one phosphate group from phytate.

Phytases are widespread in nature and have been found in bacteria, yeasts, fungi and plants and phytases from any of these may be used in the invention. The fungal enzyme phytase from Aspergillus niger has been commercialized for use in animal feed and may also be used in the present invention. The gene encoding the enzyme has been cloned and the phytase enzyme has been overexpressed in Aspergillus niger. This fungus is grown on industrial scale in large fementors allowing for the production of the enzyme. The fungus secretes considerable amounts of phytase which can be separated from the biomass in a series of filtration and ultrafiltration steps. The resulting concentrated ultrafiltrate is subsequently formulated into a stable granulate or liquid which may be used in the present invention.

The term protein arginine deiminase and peptidyl arginine deiminase (PAD) are used interchangeably herein. Protein or peptidyl arginine deiminases belong to a family of enzymes (EC 3.5.3.15) which convert peptide or protein bound arginine into peptide or protein bound citruline. This process is called deamination or citrullination. In the reaction from arginine to citruline, one of the terminal nitrogen atoms of the arginine side chain is replaced by an oxygen. The reaction uses one water molecule and yields ammonia as a side product (http://en.wikipedia.org/wiki/Citrullination). Whereas arginine is positively charged at a neutral pH, citrulline is uncharged. Surprisingly, it was found that a protein wherein at least part of the arginine has been converted into citruline, and thereby resulting in protein with less charge, exhibited modified sweetness, liquorice, astringency, powdery/chalk, fullness, thickness and/or digestibility.

Peptidyl arginine deiminase (PAD) may be derived from any suitable origin, for instance from mammalian or microbial origin. PAD's used in the present invention are advantageously derived from a microbial source, i.e. the PAD used in a process of the invention is a microbial PAD. For instance, PAD's may be derived from fungal origin such as from Fusarium sp. such as Fusarium garminearum, Chaetomium globosum, Phaesphaeria nodorum or from bacterial origin such as from the bacteria Streptomyces, eg Streptomyces scables, Streptomyces clavuligeres. The wording “derived” or “derivable” from with respect to the origin of a polypeptide as disclosed herein, means that when carrying out a BLAST search with a polypeptide as disclosed herein, the polypeptide may be derivable from a natural source, such as a microbial cell, of which an endogenous polypeptide shows the highest percentage homology or identity with the polypeptide as disclosed herein.

Peptidyl arginine deiminases are for instance known from WO2008/000714. WO2008/000714 discloses a process for enzymatically treating a protein with a protein arginine deiminase, wherein at least 30% of the arginine is transformed into citruline.

The step of “incubating a solution comprising a plant protein with phytase and a peptidyl arginine deiminase (PAD)” is performed under conditions such that phytase and PAD are able to perform their enzymatic activity. The skilled person is aware that the conditions for incubation depend for example on the specific enzyme used, enzyme concentration, time, temperature, pH etc.

In the present invention phytase is typically incorporated in an amount of at least 2 FTU per gram flour, isolate or concentrate; one FTU being the activity of phytase that generates 1 μmol of inorganic phosphorus per minute from an excess of sodium phytate at pH 5.5 and 37° C. A suitable methodology for determining phytase activity is described in M. Wyss et al.; Biophysical Characterisation of fungal phytases (myo-Inositol hexakisphosphate phosphohydrolase): Molecular size, glycosylation pattern, and engineering of proteolytic resistance. Appl. Envir. Micr., 85 (2), 359-388, 1999. Preferably, phytase is incorporated in a range of 2 to 20 FTU/g dry weight.

In the present invention PAD is typically added in an amount of 0.1% (w/w) enzyme protein to protein substrate and incubated at a suitable temperature and pH, for instance incubating a plant protein, for example pea protein, with a peptidyl arginine deiminase at a pH of between 4 and 9, such as a pH of between 5 and 8.5, such as a pH of between 5.5 and 8, such as a pH between 8 and 7, or a pH of between 6.2 and 6.8, for instance at a pH of about 6.5. A suitable temperature at which protein is incubated with PAD may be between 20 and 60 degrees Celsius, such as a between 30 and 50, or between 35 and 45 degrees Celsius. Peptidylarginine deiminase activity was determined by measuring the formation of citruline residues in α-N-Benzoyl-L-arginine-ethyl ester (BAEE). The incubation mixture contained 100 mM tris-HCl buffer (pH 7.5), 5 mM CaCl₂, 10 mM DTT, 10 mM BAEE in a final volume of 700 μl. Incubation was performed at 55° C. for 30 min, and the reaction was stopped by adding 100 μl 8 N HClO₄. Citrulline was determined by colorimetry according to the method of Guthöhrein and Knappe, (1968) Anal. Biochem. 26, 188. One unit of peptidyl arginine deiminase is expressed as 1 μmol of citruline formed/min/mg of protein.

PAD and phytase are added to a solution comprising a plant protein and subsequently incubated such as to obtain a phytase and PAD-treated solution. i.e. the invention provides a method for preparing a plant protein comprising drink with an improved property, said method comprising
incubating a solution comprising a plant protein with phytase and a peptidyl arginine deiminase (PAD) to obtain a phytase and PAD treated solution and

optionally processing the phytase and PAD treated solution into a drink comprising a plant protein, wherein the plant protein is pea protein or faba bean protein.

Preferably, the plant protein is pea protein.

The step of “incubating a solution comprising a plant protein with phytase and a PAD” results in a phytase and PAD treated solution. The phytase and PAD treated solution can be subjected to an enzyme (i.e. phytase and PAD) inactivation step, for example by heating the treated solution to 90° C. and keeping at 90° C. for 15 minutes. The enzyme inactivation step can also be combined with a heat treatment step to reduce the amount of micro-organisms.

The phytase and PAD treated solution can be the final food product.

A method as described above may comprise an optional step of processing the phytase and PAD treated solution into a final product, i.e. a drink comprising plant protein. An example of a step wherein the phytase and PAD treated solution is processed into a drink comprising plant protein is homogenisation.

A method of the invention may also comprise a step of adding an ingredient, such as
adding of a sugar such as cane sugar,
adding of a micronutrient such as a vitamin or a mineral (for example calcium),
adding of (reduced amounts of) a stabilizer or a hydrocolloid,
adding of ol,
adding of (reduced amounts of) flavour components.

A method of the invention can further comprise a step of packaging the drink into a suitable container.

In case a pea flour is used, a preferred additional step is: incubating a solution comprising a pea protein with alpha-amylase.

In one its embodiment, the invention provides a method as described herein wherein said method excludes the addition of a hydrocolloid or a stabilizer such that a clean label product can be prepared.

Examples of a plant-based drink are a plant-based milk, a fortified plant-based drink, a plant-based drink with a neutral or acidic pH or a sport plant-based drink. Yet another example of a plant-based drink is a daily shake alternative. The fortified plant-based drink is for example a protein fortified plant-based drink or a vitamin fortified plant-based drink.

The property which is preferably improved in a food product comprising a plant protein is texture, taste, mouthfeel or viscosity.

The term “mouthfeel” refers to the physical sensation in the mouth caused by food or drink and is distinct from taste. Mouthfeel is a fundamental sensory attribute which, along with taste and smell, determine the overall flavour of a food product.

The term “taste” refers to the perception produced or stimulated when a substance in the mouth reacts chemically with taste receptor cells located on taste buds in the oral cavity, mostly on the tongue.

The term “texture” refers to visual or mechanical assessment of a food product.

Examples of a property are smoothness, astringency, thickness or, in pea-based drinks, pea flavour.

An example of a texture property is smoothness.

An example of a mouthfeel property is smoothness.

Examples of a taste property are astringency or pea taste.

It is also possible to have combined properties, for example texture and mouthfeel. An example of texture and mouthfeel (i.e. combined properties) is smoothness.

Another example of a property is viscosity.

Whether or not a property is improved can be easily determined by comparing a method of the invention with a similar method in which phytase and PAD are not used but al other conditions are identical. An improvement in one or more of the properties related to texture, taste or mouthfeel may be measured absolutely for instance in the case of Brookfield (Pa*s units) or shear stress (Pa units) or more relatively by a taste panel for instance for al the sensory aspects of the plant-based drink. The improvement of a property (such as decreased astringency, increased smoothness, reduced pea flavour, reduced thickness or reduced viscosity) is at least 2%, 3%, 5%, 10%, for instance at least 20%, 25%, 30%, 35%, 40%, 45%, 50%, 55%, 60%, 65%, 70%, 75%, 80%, 85% or at least 90% or 95%. The improvement of a property of a plant protein comprising drink that has been incubated with phytase and PAD is compared to a plant protein comprising drink that has not been incubated with phytase and PAD under the same conditions.

I.e. the invention provides a method as described herein, wherein the drink has an improved property as compared to a drink comprising a plant protein that has not been prepared in the presence of phytase but which drink has otherwise been prepared identically.

The invention provides a method for preparing a plant protein drink with an improved property, for example a pea protein or a faba bean protein drink.

Preferably, the invention provides a method for preparing a pea protein comprising drink with an improved property, said method comprising
incubating a solution comprising a pea protein with phytase and a peptidyl arginine deiminase (PAD) (to obtain a phytase & PAD-treated solution) and

optionally processing the phytase and PAD treated solution into a drink comprising a pea protein,
wherein said improved property is decreased astringency, increased smoothness, reduced pea flavour, reduced thickness or reduced viscosity. Preferably, said improved property is reduced pea flavour or reduced viscosity. Also preferred, is a method as described herein wherein at least two properties are improved, preferably reduced pea taste and reduced viscosity.

The term “pea flavour” as used herein refers to the typical earthy or grass taste and smell of pea products.

The term “smoothness” as used herein refers to the degree in which the product is smooth without grittiness, grains and lumps. This can for example be judged by lifting a spoon and assess the back of the spoon.

The term “astringency” as used herein refers to the dry, puckering mouthfeel similar to that caused by tannins that are for example found in many fruits or by proteins, especially in an acidic environment. Can for example be determined by the degree in which an astringent afterfeel is present in the mouth after the product is swallowed.

The term “thickness” as used herein refers to viscosity perceived in the mouth

The term “mouthfeel” refers to the texture perceived in the mouth, a physical sensations in the mouth caused by food or drink which can be distinguished from the measured viscosity, a physical measured property.

The term “viscosity” refers to the state of being thick, sticky, and semi-fluid in consistency, due to internal friction which is measured by the force per unit area resisting uniform flow.

Phytase and PAD can be added sequentially to a solution comprising plant protein or simultaneously. Surprisingly, the best improvements are obtained when said solution is first incubated with PAD and subsequently with phytase. The invention thus provides a method for preparing a plant protein comprising drink with an improved property, said method comprising
incubating a solution comprising a plant protein with phytase and a peptidyl arginine deiminase (PAD) (to obtain a phytase & PAD-treated solution) and

optionally processing the phytase and PAD treated solution into a drink comprising a plant protein wherein the plant protein is pea protein or faba bean protein,

wherein said solution is first incubated with PAD and subsequently with phytase.

Preferably, the plant protein is pea protein.

After addition of PAD, PAD is allowed to perform its enzymatic activity under certain conditions (for example 2 hours at 45 degrees Celsius) and subsequently phytase is added which is also allowed to perform its enzymatic activity for example 2 hours at the same temperature.

In yet another aspect, the invention provides a drink obtainable by a method of the invention, i.e. by a method for preparing a plant protein comprising drink with an improved property, said method comprising
incubating a solution comprising a plant protein with phytase and a peptidyl arginine deiminase (PAD) and

optionally processing the phytase and PAD treated solution into a drink comprising a plant protein, wherein the plant protein is pea protein or faba bean protein.

Preferably, the plant protein is pea protein.

Examples of a drink have been described above and apply to this part of the invention as well. A plant-based drink of the invention can be distinguished from a prior art plant-based drink in view of the (i) phytate content or (ii) reduced levels of thickeners or stabilizers or (iii) absence of thickeners or stabilizers or (iv) citrulline content.

In a further aspect the invention provides use of phytase and peptidyl arginine deiminase (PAD) for improving texture, taste or mouthfeel or viscosity of a drink comprising a plant protein.

The plant protein may be, for example, pea protein or faba bean protein. Preferably, the plant protein is pea protein.

In one of its aspects, the invention provides use of phytase and PAD for decreasing astringency, increasing smoothness, reducing thickness of a drink comprising a plant protein, wherein the plant protein is pea protein or faba bean protein.

When the plant protein is pea protein, the invention provides use of phytase and PAD for decreasing astringency, increasing smoothness, reducing pea flavour, reducing thickness of a drink comprising a pea protein.

The definitions and explanations which are given above for the method claims apply to the use claims as well.

The invention will be explained in more detail in the following example with respect to pea protein sources (pea flour and pea protein isolate), which are not limiting the invention. The pea protein sources may be easily substituted by proteins sourced from faba bean.

### EXPERIMENTAL PART

### Example 1

Phytic Acid Content in Pea Materials Treated with Phytase

The pea protein ingredients (pea flour and pea protein isolate) were received from Roquette Freres S.A. 15% Dry matter pea flour or 5% dry matter pea isolate was adjusted to pH 6.5 with phosphoric acid and incubated with phytase enzyme at 50° C. for up to 30 min at an enzyme dose of 20 FTU/g solids. Phytate analysis of samples was performed using 31P-NMR. Of each sample 2 g was mixed with 12.5 ml 0.5 M HCl, mixed thoroughly and put in a sonication bath (Branson 5510) for 30 minutes. Samples of 2×2 ml of each suspension were transferred into Eppendorf centrifuge tubes and centrifuged during 15 min at 17949 relative centrifugal force (rcf). 2000 μl of the supernatants and 500 μl of an internal Standard solution were mixed (standard solution contains: +/−25 mg phosphonoacetic acid, 2.25 g EDTA, 6.5 ml deuterated water and 2.5 ml 6 N KOH in a 25.0 ml volumetric flask, replete with demineralized water) and pH was set to pH 7.5. After lyophilization the residues were resuspended in 1 ml of deuterated water. The pH of the solutions was adjusted to pH 12.5 prior to the NMR measurements. 31P-spectra were recorded at 400 MHz on a Bruker NMR spectrometer equipped with a nitrogen cooled cryoprobe.

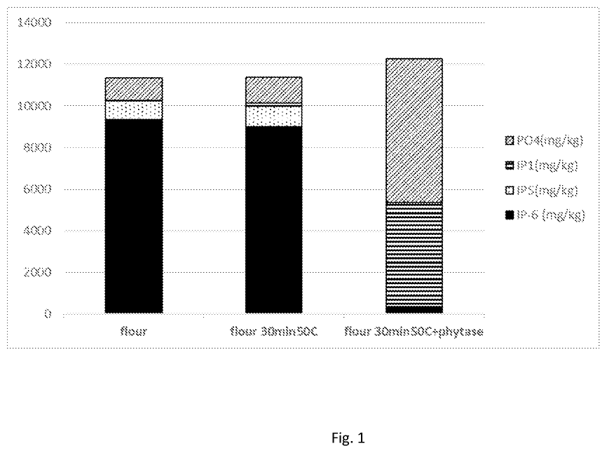

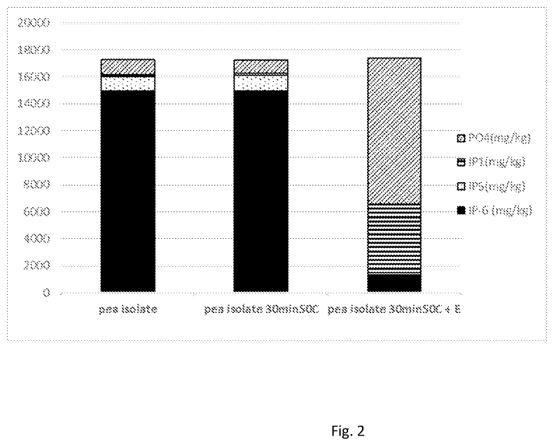

The results are shown in FIG. 1 (pea flour) and FIG. 2 (pea isolate).

Conclusion FIG. 1: Almost complete conversion of phytic acid (IP6) to IP1 was obtained with the phytase treatment in these conditions. No endogenous phytase activity present in the flour.

Conclusion FIG. 2: More than 90% conversion of the phytic acid to IP1 was obtained in these conditions with the phytase treatment. No endogenous phytase activity present in the pea isolate.

### Example 2

Sensory Analysis of Pea Beverages

The pea protein ingredients (pea flour and pea protein isolate) were received from Roquette Freres S.A. The pea beverages were produced at 2 L scale in a food grade environment. The process of making the pea beverage from pea protein isolate consists of the pea ingredient dispersion (6% total solids) for 1 hour at 45° C. and adjustment of the pH to pH 6.5 followed by incubation with PAD enzyme at a dose of 0.2 g enzyme per kg of protein isolate for 2 hours. After this phytase is added at a dose of 20 FTU/g solid an incubated further at 45° C. for 2 hours. After enzyme incubation the wet ingredients were added to make a final beverage recipe as shown in Table 1 below. The process of making the beverage from pea flour was similar. An additional step was added after dispersion of the pea flour (12.5% total solids) which involves incubation with alpha-amylase at 75° C. for 1 hour in order to decrease the viscosity of the beverage. The beverages were further homogenized (using a pressure homogenizer 250/50 bar) and then heated to 95° C. for 15 min to deactivate the enzymes.

Two recipes were defined depending on the pea-containing starting material that was used (Table 1). Recipe 1 based on pea flour contained ⅔ of cow's milk protein and fat content, while recipe 2 based on pea isolate had same composition than milk regarding macronutrients (Table 2).

TABLE 1
Ingredient list and proximal composition of the pea beverages

| | from pea | from pea |
|---|---|---|
| | flour beverage, | isolate beverage, |
| Ingredient | % w/w | % w/w |
| Pea source | 9.9 | 4.7 |
| Cane sugar | 3.25 | 3.25 |
| High oleic sunflower oil | 2.6 | 2.6 |
| Tricalcium phosphate | 0.35 | 0.35 |
| Phosphate salt | <0.5 | <0.5 |
| Gellan gum | 0.115 | 0.115 |
| Phosphoric acid | n.a. | <0.1 |

TABLE 2
Proximal composition of the pea beverages

| | From pea | From pea |
|---|---|---|
| Nutrient | flour, % w/w | isolate, % w/w |
| Protein | 2 | 3.4 |
| Fat | 2.8 | 3.0 |
| Carbohydrates | 9.4 | 3.4 |

The beverages produced from pea flour and pea isolate with and without enzyme treatment were analyzed by sensory panel (N=5) for sensory attribute comparisons.

TABLE 3
Sensory analysis of the pea beverages

| | Texture/ | |
|---|---|---|
| Sample | mouthfeel | Taste and flavor |
| Pea flour beverage + | Thick, | Sweet, grass, astringent, |
| amylase | chalk/grainy | pea notes |
| Pea flour beverage + | More mouthfeel, | Less astringent, neutral |
| amylase with PAD and | smooth | taste, less pea notes |
| phytase | | |
| Pea isolate beverage | Thick | Bitter, astringent, pea notes |
| Pea isolate beverage | Less thick | Not astringent, neutral |
| with PAD and phytase | | taste, less pea notes |

### Example 3

Viscosity of the Pea Beverages Treated with Enzymes

The two types of pea beverages (flour-based or isolate-based) were prepared as described above in example 2. Beside the combination of PAD and phytase enzyme used in the process, the single enzymes, either PAD or phytase were used to prepare the two types of drinks. The pea beverages were incubated under the same conditions and enzyme dosages as described for the combination of the two enzymes. PAD enzyme was used at a dose of 0.2 g enzyme per kg of pea protein, while phytase was added at 20 FTU/g sold.

Viscosity of the Samples was Measured with the Brookfield RVDV-II+Pro Viscometer.

The viscosity of the pea beverage samples was measured using spindle RV-3 (measurement without Helipath). At 21 points in time (3 sec interval) viscosity was measured at speed 30 rpm. Before starting, the spindle was lowered into the samples up until the spindle shaft indentation. For each pea beverage the sample was poured in one 125 ml cup and measured. The final viscosity was defined as the average viscosity over the final 30 seconds of the measurement (10 datapoints).

The measured viscosity was lower in the PAD alone process compared to the no enzyme treated pea beverages. The viscosity was increased in the pea beverages processed with phytase alone with significantly more increase in viscosity in the isolate-based pea beverage compared to the flour-based beverage compared to the non-enzyme process. The combination of PAD and phytase showed a lower viscosity than the non-enzyme treated beverages.

“The activity leading to this application has received funding from the European Institute of Innovation and Technology (EIT), reference number 2.2.16 under grant agreement No [19177]. This European body receives support from the European Union's the Horizon 2020 research and innovation programme”

1. A method for preparing a plant protein comprising a drink with an improved property, said method comprising
incubating a solution comprising a plant protein with phytase and a peptidyl arginine deiminase (PAD) and
optionally processing the phytase and PAD treated solution into a drink comprising a plant protein,
wherein the plant protein is pea protein or faba bean protein.

2. The method according to claim 1, wherein the plant protein is pea protein.

3. The method according to claim 1, wherein said improved property is improved texture, taste, mouthfeel or viscosity.

4. The method according to claim 1, wherein said improved property is decreased astringency, increased smoothness, reduced thickness or reduced viscosity.

5. A method according to claim 1, wherein said improved property is reduced pea flavour.

6. The method according to claim 1, further comprising dissolving flour, plant protein isolate powder or plant protein concentrate powder to obtain a solution comprising a plant protein.

7. The method according to claim 1, wherein said solution is first incubated with PAD and subsequently with phytase.

8. The method according to claim 1, wherein said drink is a fortified drink.

9. The method according to claim 1, wherein said drink is a drink with a neutral or acidic pH.

10. A drink comprising a plant protein obtainable by a method according to claim 1, wherein the plant protein is pea protein or faba bean protein.

11. The drink according to claim 10, wherein the plant protein is pea protein.

12. A product comprising phytase and PAD for improving texture, taste, mouthfeel and/or viscosity of a drink comprising a plant protein, wherein the plant protein is pea protein or faba bean protein.

13. The product according to claim 12, wherein the plant protein is pea protein.
